SEQUENCE LISTING (22 sequences)

SEQ ID NO: 1 - DNA, 3812 bp, Peanut (Arachis hypogaea L.)
tggtcctgaaattaaaacattatcgtttaatttatacttgtgtcattccctccaagacttctttcctctatttatttattttttgaatataaagattaaagaacatgagcaccacaaactttttaaagataagaaaagaaaaataaaaaataaaagtgacaagtgacctttaagagtattataaatatggatgtagagatcacactcccattatacccaacaacaaacaaaattcacttcatattcttcttcttctctttcattctctttaacaatggcaaagatgtcatcttcagatcctttagttcttggaagagtggttggagatgtcatccactctttcaatccaagtgttcaaatgtctgtcacttacaacaccaaacaagtcttcaatggccatgagttcttcccttctgctcttaccactaggcctaaggttgccattgatggtggtgacatgaggactttttacacactggtaataatttcattattcatacatatatatatatattatttgttataatagaagagaggcaaaaaataattaataaataatattatggtagatattaaaaaaaaaataagggtgatcattgttgatgattgtgagtttgcttttttttttcctatttaattttacactagatcaattttacttacaagatttcactactatttaatttgttgatcctttttctctcccaaaaaagggggaaaaacaaaaccctaggggttagttcttacaaaatttggagctatttgagaaaaattaatgtatcctcttgtttcttttgtagatcatgacagatcctgatgttcctggcccaagtgatccttatctgagagaacatttgcactggtaacttgatatatatgattgaatttgaacctaggttttttattttttttttattttatttttttctagtaaaattaggtttaattatataactttcatgtaatacaaaagaaaaatgtctgtttcatcatatatactaaaagttgaccttcaaaaacaaattatgagaagagaaatagtgacaataatcttatgattaatacatgaactttggtaaagttatactaaacctttttttggctctcaaatctaactagtgtatttagaacttattagccttttcacttttgaatacaaattttcttaatttaattaaagcattagctatagataaatccccaatatgtttaatttatccactaaaaatagtgatatcacatgaactatcttataattaagaagaagcctttttcccctctcttacacacacactctcttttttttcctcctaatattcacattttaaacaaatactaatataattcttgtacaattattgtaaatacatatacttggtatattacctctaacatatatatctatttatagcaattatacaaaaaattatacacaataattaattatttatataacatttttgcacattttaatacacaaaaaataattaagtgtagatagagaggcttctgatctaacagtctactcttaattggttgcttttgcaggatggttacagacattcctggcacaacagatgccacatttggtaggtttcattgattcatagatctaattaaaatacaccatatatgtgttattctgtgttattcatttctatatataattcaaattgacctaccttattattattaagtgaaaatgttttatacattatatagactacccaactatcaaatgactatactaattttttttttatgtatctttcaaaaataaaatagttacaccaaatatttgcatgaactgaagtctagaatttagttcatattgtactatattatgaatacaattactaaaattatctactaataaaatgacaaagattcacatgtagttatttttatgtcaattatagttaatagttaaaaatcgttaaataataatttagttaaattaatggttcttaaatattaacatcatgtgagtttttaccggataaaatatatatatttatatataaatacatagtgactgattttaataattaatatatgtctgataagtaataaaaaagataaatgaataatagtaacaattttgtttgatgtaaaatggaagggaaagagatagtgagctatgaaagccctaagccacagatagggatacacaggtatgtgtttgtgttgttgaagcaaaagagaaggcagagtgtaaccccaccaagttcaagggatcacttcaacactatcaacttctctgctcactctgacctttctcttcctgttgctgctgtctacttcaatgctcagagagaaaccgccgctagaagacgctaattcatatatatgtagctgcatgcacacctatatatatatatatttattattagtaaataaagcaagaagcatgcgagcaaggttaggttatggctatatatcatcatcatcatcatgaagaatgaaaagggtatggcacgtggcttcttctcataggtggtgttaatcttggatatatagtttaggtgtcacttattattatatcatgtatgtggctaattaatgtattgtgttagttgtatgtgttgtttgtgagttatgtaatataatgttatgaacttgagaattaagaataaataaatatctttttattactacagtaacctagtttaatttccctatatatatatatatatatatatatatatatatatatatatatatatatatatatatatatatatatatatatatataacaataaaaaatagtcatgtttacaagccaagaaatgatagaaattaataacaataataatgaacttattagaaaatatttaagatttgatcgatcatttattcatgaaaaaatataggaaactaagtttcagataattaatattgtttaatgttttttattgtaaaattttttctttaacctttattttgaaaaatttaggatttagaattaaaatataacatataaaattaaggatttaaaatttaaaattaaaaaaattaactgatcttggctaaaaaaattagctccttagttgaactcattcatgcatcctaattgatgcttttagtttcgtttattgttaggaatgaaataaatagacataggtgtgtttgcaattattataaccaattttctaattttcaagcttaaaataatatatatatttttttcatataatcacagatgttcgtgtatcatttaactgccataactttttagtttcaactttcagcattcaaattaagccaaaatgtaatatgcattagttaggttattagtgtgagagaaaactcaaaatttgagttataataataataacaattgataaatcataaagttcaatctgttgttaaagaaaattcagcaacactcatatataagggtatgttatgtattataaaataaatcattttggaattgttatatatattaattaatattaatacaaaagatcgaatacatttttatcaaaaagttcaatttctgtttatttttccgaaagatgatatgacttctaacaaaaataaatcacaaaggctcttaattattcactttttgagacaatttgatcttcttctttttttttttttacctaaaacaatgttatattgaggaattgaatagtttgtattttaataaaaacccgatgtaatttggaattaaaaatatcggggatgtcttaaacaaggtaaataattaaaaaaaaattcagtttatttaacaaaaataaatattaatgattattttatttattttaaatttataagattaaactatctgatattaaaaat

SEQ ID NO: 2 - DNA, 1116 bp, Peanut (Arachis hypogaea L.)
tggtcctgaaattaaaacattatcgtttaatttatacttgtgtcattccctccaagacttctttcctctatttatttattttttgaatataaagattaaagaacatgagcaccacaaactttttaaagataagaaaagaaaaataaaaaataaaagtgacaagtgacctttaagagtattataaatatggatgtagagatcacactcccattatacccaacaacaaacaaaattcacttcatattcttcttcttctctttcattctctttaacaatggcaaagatgtcatcttcagatcctttagttcttggaagagtggttggagatgtcatccactctttcaatccaagtgttcaaatgtctgtcacttacaacaccaaacaagtcttcaatggccatgagttcttcccttctgctcttaccactaggcctaaggttgccattgatggtggtgacatgaggactttttacacactgatcatgacagatcctgatgttcctggcccaagtgatccttatctgagagaacatttgcactggatggttacagacattcctggcacaacagatgccacatttgggaaagagatagtgagctatgaaagccctaagccacagatagggatacacaggtatgtgtttgtgttgttgaagcaaaagagaaggcagagtgtaaccccaccaagttcaagggatcacttcaacactatcaacttctctgctcactctgacctttctcttcctgttgctgctgtctacttcaatgctcagagagaaaccgccgctagaagacgctaattcatatatatgtagctgcatgcacacctatatatatatatatttattattagtaaataaagcaagaagcatgcgagcaaggttaggttatggctatatatcatcatcatcatcatgaagaatgaaaagggtatggcacgtggcttcttctcataggtggtgttaatcttggatatatagtttaggtgtcacttattattatatcatgtatgtggctaattaatgtattgtgttagttgtatgtgttgtttgtgagttatgtaatataatgttatgaacttgagaattaagaataaataaatatctttttattacta

SEQ ID NO: 3 - protein, 174 aa, Peanut (Arachis hypogaea L.)
MAKMSSSDPLVLGRVVGDVIHSFNPSVQMSVTYNTKQVFNGHEFFPSALTTRPKVAIDGGDMRTFYTLIMTDPDVPGPSDPYLREHLHWMVTDIPGTTDATFGKEIVSYESPKPQIGIHRYVFVLLKQKRRQSVTPPSSRDHFNTINFSAHSDLSLPVAAVYFNAQRETAARRR

SEQ ID NO: 4 - DNA, 25 bp, Artificial Sequence
  Sequence is synthesized
atggatgtagagatcacactcccat

SEQ ID NO: 5 - DNA, 20 bp, Artificial Sequence
  Sequence is synthesized
tagccataacctaaccttgc

SEQ ID NO: 6 - DNA, 25 bp, Artificial Sequence
  Sequence is synthesized
aacaaaattcacttcatattcttct

SEQ ID NO: 7 - DNA, 20 bp, Artificial Sequence
  Sequence is synthesized
cataacctaaccttgctcgc

SEQ ID NO: 8 - DNA, 2666 bp, Peanut (Arachis hypogaea L.)
tggtcctgaaattaaaacattatcgtttaatttatacttgtgtcattccctccaagacttctttcctctatttatttattttttgaatataaagattaaagaacatgagcaccacaaactttttaaagataagaaaagaaaaataaaaaataaaagtgacaagtgacctttaagagtattataaataggtggatactacaatgaagatggcataattgtcttcatatgagtatatttctttttgacctttggatgatggattgtatggttagattttgatatttataaatgtgttgtttttgtttaaagtgtggctaaataaataaaccacacttttaactaaagcatcttcatgagaagatatttttgccatcttcattgtagtatccaccaaataaatatggatgtagagatcacactcccattatacccaacaacaaacaaaattcacttcatattcttcttcttctctttcattctctttaacaatggcaaagatgtcatcttcagatcctttagttcttggaagagtggttggagatgtcatccactctttcaatccaagtgttcaaatgtctgtcacttacaacaccaaacaagtcttcaatggccatgagttcttcccttctgctcttaccactaggcctaaggttgccattgatggtggtgacatgaggactttttacacactggtaataatttcattattcatacatatatatatatattatttgttataatagaagagaggcaaaaaataattaataaataatattatggtagatattaaaaaaaaaataagggtgatcattgttgatgattgtgagtttgcttttttttttcctatttaattttacactagatcaattttacttacaagatttcactactatttaatttgttgatcctttttctctcccaaaaaagggggaaaaacaaaaccctaggggttagttcttacaaaatttggagctatttgagaaaaattaatgtatcctcttgtttcttttgtagatcatgacagatcctgatgttcctggcccaagtgatccttatctgagagaacatttgcactggtaacttgatatatatgattgaatttgaacctaggttttttattttttttttattttatttttttctagtaaaattaggtttaattatataactttcatgtaatacaaaagaaaaatgtctgtttcatcatatatactaaaagttgaccttcaaaaacaaattatgagaagagaaatagtgacaataatcttatgattaatacatgaactttggtaaagttatactaaacctttttttggctctcaaatctaactagtgtatttagaacttattagccttttcacttttgaatacaaattttcttaatttaattaaagcattagctatagataaatccccaatatgtttaatttatccactaaaaatagtgatatcacatgaactatcttataattaagaagaagcctttttcccctctcttacacacacactctcttttttttcctcctaatattcacattttaaacaaatactaatataattcttgtacaattattgtaaatacatatacttggtatattacctctaacatatatatctatttatagcaattatacaaaaaattatacacaataattaattatttatataacatttttgcacattttaatacacaaaaaataattaagtgtagatagagaggcttctgatctaacagtctactcttaattggttgcttttgcaggatggttacagacattcctggcacaacagatgccacatttggtaggtttcattgattcatagatctaattaaaatacaccatatatgtgttattctgtgttattcatttctatatataattcaaattgacctaccttattattattaagtgaaaatgttttatacattatatagactacccaactatcaaatgactatactaattttttttttatgtatctttcaaaaataaaatagttacaccaaatatttgcatgaactgaagtctagaatttagttcatattgtactatattatgaatacaattactaaaattatctactaataaaatgacaaagattcacatgtagttatttttatgtcaattatagttaatagttaaaaatcgttaaataataatttagttaaattaatggttcttaaatattaacatcatgtgagtttttaccgaataaaatatatatatttatatataaatacatagtgactgattttaataattaatatatgtctgataagtaataaaaaagataaatgaataatagtaacaattttgtttgatgtaaaatggaagggaaagagatagtgagctatgaattgaatagtttgtattttaataaaaacccgatgtaatttggaattaaaaatatcggggatgtcttaaacaaggtaaataattaaaaaaaaattcagtttatttaacaaaaataaatattaatgattattttatttattttaaatttataagattaaactatctgatattaaaaatttttaagaataattaaatttgagtaattattcattacttttttctcttgtgatgtcaattagtgaataatgcatgaatttattttaaaatacgagactataagaaaaaaaactgaaatagtttattttttaa

SEQ ID NO: 9 - DNA, 24 bp, Artificial sequence
  Sequence is synthesized
aacttattagccttttcacttttg

SEQ ID NO: 10 - DNA, 23 bp, Artificial sequence
  Sequence is synthesized
ttaattccaaattacatcgggtt

SEQ ID NO: 11 - DNA, 2654 bp, Peanut (Arachis hypogaea L.)
tggtcctgaaattaaaacattatcgtttaatttatacttgtgtcattccctccaagacttctttcctctatttatttattttttgaatataaagattaaagaacatgagcaccacaaactttttaaagataagaaaagaaaaataaaaaataaaagtgacaagtgacctttaagagtattataaatatggatgtagagatcacactcccattatacccaacaacaaacaaaattcacttcatattcttcttcttctctttcattctctttaacaatggcaaagatgtcatcttcagatcctttagttcttggaagagtggttggagatgtcatccactctttcaatccaagtgttcaaatgtctgtcacttacaacaccaaacaagtcttcaatggccatgagttcttcccttctgctcttaccactaggcctaaggttgccattgatggtggtgacatgaggactttttacacactggtaataatttcattattcatacatatatatatatattatttgttataatagaagagaggcaaaaaataattaataaataatattatggtagatattaaaaaaaaaataagggtgatcattgttgatgattgtgagtttgcttttttttttcctatttaattttacactagatcaattttacttacaagatttcactactatttaatttgttgatcctttttctctcccaaaaaagggggaaaaacaaaaccctaggggttagttcttacaaaatttggagctatttgagaaaaattaatgtatcctcttgtttcttttgtagatcatgacagatcctgatgttcctggcccaagtgatccttatctgagagaacatttgcactggtaacttgatatatatgattgaatttgaacctaggttttttattttttttttattttatttttttctagaaaaattaggtttaattatataactttcatgtaatacaaaagaaaaatgtctgtttcatcatatatactaaaagttgaccttcaaaaacaaattatgagaagagaaatagtgacaataatcttatgattaatacatgaactttggtaaagttatactaaacctttttttggctctcaaatctaactagtgtatttagaacttattagccttttcacttttgaatacaaattttcttaatttaattaaagcattagctatagataaatccccaatatgtttaatttatccactaaaaatagtgatatcacatgaactatcttataattaagaagaagcctttttcccctctcttacacacacactctcttttttttcctcctaatattcacattttaaacaaatactaatataattcttgtacaattattgtaaatacatatacttggtatattacctctaacatatatatctatttatagcaattatacaaaaaattatacacaataattaattatttatataacatttttgcacattttaatacacaaaaaataattaagtgtagatagagaggcttctgatctaacagtctactcttaattggttgcttttgcaggatggttacagacattcctggcacaacagatgccacatttggtaggtttcattgattcatagatctaattaaaatacaccatatatgtgttattctgtgttattcatttctatatataattcaaattgacctaccttattattattaagtgaaaatgttttatacattatatagactacccaactatcaaatgactatactaattttttttttatgtatctttcaaaaataaaatagttacaccaaatatttgcatgaactgaagtctagaatttagttcatattgtactatattatgaatacaattactaaaattatctactaataaaatgacaaagattcacatgtagttatttttatgtcaattatagttaatagttaaaaatcgttaaataataatttagttaaattaatggttcttaaatattaacatcatgtgagtttttaccggataaaatatatatatttatatataaatacatagtgactgattttaataattaatatatgtctgataagtaataaaaaagataaatgaataatagtaacaattttgtttgatgtaaaatggaagggaaagagatagtgagctatgaaagcctaagccacagatagggatacacaggtatgtgtttgtgttgttgaagcaaaagagaaggcagagtgtaaccccaccaagttcaagggatcacttcaacactatcaacttctctgctcactctgacctttctcttcctgttgctgctgtctacttcaatgctcagagagaaaccgccgctagaagacgctaattcatatatatgtagctgcatgcacacctatatatatatatatttattattagtaaataaagcaagaagcatgcgagcaaggttaggttatggctatatatcatcatcatcatcatgaagaatgaaaagggtatggcacgtggcttcttctcataggtggtgttaatcttggatatatagtttaggtgtcacttattattatatcatgtatgtggctaattaatgtattgtgttagttgtatgtgttgtttgtgagttatgtaatataatgttatgaacttgagaattaagaataaataaatatctttttattac

SEQ ID NO: 12 - DNA, 1105 bp, Peanut (Arachis hypogaea L.)
attaaaacattatcgtttaatttatacttgtgtcattccctccaagacttctttcctctatttatttattttttgaatataaagattaaagaacatgagcaccacaaactttttaaagataagaaaagaaaaataaaaaataaaagtgacaagtgacctttaagagtattataaatatggatgtagagatcacactcccattatacccaacaacaaacaaaattcacttcatattcttcttcttctctttcattctctttaacaatggcaaagatgtcatcttcagatcctttagttcttggaagagtggttggagatgtcatccactctttcaatccaagtgttcaaatgtctgtcacttacaacaccaaacaagtcttcaatggccatgagttcttcccttctgctcttaccactaggcctaaggttgccattgatggtggtgacatgaggactttttacacactgatcatgacagatcctgatgttcctggcccaagtgatccttatctgagagaacatttgcactggatggttacagacattcctggcacaacagatgccacatttgggaaagagatagtgagctatgaaagcctaagccacagatagggatacacaggtatgtgtttgtgttgttgaagcaaaagagaaggcagagtgtaaccccaccaagttcaagggatcacttcaacactatcaacttctctgctcactctgacctttctcttcctgttgctgctgtctacttcaatgctcagagagaaaccgccgctagaagacgctaattcatatatatgtagctgcatgcacacctatatatatatatatttattattagtaaataaagcaagaagcatgcgagcaaggttaggttatggctatatatcatcatcatcatcatgaagaatgaaaagggtatggcacgtggcttcttctcataggtggtgttaatcttggatatatagtttaggtgtcacttattattatatcatgtatgtggctaattaatgtattgtgttagttgtatgtgttgtttgtgagttatgtaatataatgttatgaacttgagaattaagaataaataaatatctttttattacta

SEQ ID NO: 13 - DNA, 2256 bp, Peanut (Arachis hypogaea L.)
aaccaccttcttgaaaagctctgttgcctaattaattagtcttaattaaatttcaccatgtcaactaagcatttaaaatttaaaacaatttaaattgatcgaataattaacttatttatttatttatgtaaatattaagattcaaattttgttttatatatataataatttattgatttaatgacaaattcttaaataaaatttaaatctatttatgataaattatttttacgataatactagaaaaaaaacaattagaacatactttatttaatatttattaattattgcaacaattgataaatactaaataatataaatttttactgtatttgactaattttttttattactgaatatttttgttatttttacttacgttaccatagtttgcatgcgcacacacatgtatatatatggtgaaaactcaagtgaagttgatacctgagagccgttaaatgatttgactgatttgattagattttcatccaacaactcttaggtatcaactttacgtaaagtcgacttcgcctgagttttcactcatatatatacttgctaagcattattaattttttttcatttttatatatatacactttccatataattttgcgcacttgttttttcttttttgacatgatatacataggtacattttgagaaaaataaggagttttgattagtgtatataaaagaggccttcgtcgtatgcaaagaatatatatatgtgagcgagccaacatgtaaacagtggtgaatgacaaacttttttcaaagaaaagagagaggacccgcatggcaatagcacgcaaattaaataatgatgccttcaattctgatttctgagtacaaaagaaaaattagccaattaataagaaccaattcagatggagacaatattgtaccttgtgagaattagagggatatgatttatttgcgtcataaataacaacatcttcaacttatatatattaaagtttatacacgaaatctcaacttgtttacgtaataatgtattaattatttatatattttatcaaatatattaaattattaaaatcattttaaacttgaaagatcacttttgtttgagttattttaaagaaagaatgtatcttttattgaaaaattttaaataaattaaaataaaataaaataacttctattttaaaaaattaaatttaaaatttaaattcattataaaataataaactcaacaaaaaatcaatatttttaataagtaaaaagggtcattatgaaaataataataaatcctatatgataaaattgaagtgtgaaaaaagaaaatctttgaagaagaagacgatgatgatatcatttaggttgtaaagagtaaaaagatgaaaaaagatatttgtggtggtgacgatgatagcaataataaattaataataatagcaaattaaatagtaacttaaaaaaaatttcaaaaaatgaagagataagaaaaaaaaaacataaaaaattcagaagggttttagtggcattttatttataattgatcaacgtcttattagttttgtcaaatatattgtattttttatgaatattacattagtttaatttattatagttaaatttgtcagaaataaaatttttcatggttaaaacttagaaacggtaatttatctatttattcatgcactactaatctactagctagtatcctttttagcaaaagtttcttttcttgtagcttcatttgtttaaatagtttgtttttcctatagatagttttttttttgttaccatcactagatagagaaaatataaatattgtaaacagattaaaaaaataaatattcaaaaaaagcagataaacaacttaaaattattattattaaaattaataatataaaaaagtctttattattattattattattattattattattattattattattattattattattattattattattattattattattattattattatttggtcctgaaattaaaacattatcgtttaatttatacttgtgtcattccctccaagacttctttcctctatttatttattttttgaatataaagattaaagaacatgagcaccacaaactttttaaagataagaaaagaaaaataaaaaataaaagtgacaagtgacctttaagagtattataaatatggatgtagagatcacactcccattatacccaacaacaaacaaaattcacttcatattcttcttcttctctttcattctctttaaca

SEQ ID NO: 14 - DNA, 2479 bp, Peanut (Arachis hypogaea L.)
aaccaccttcttgaaaagctctgttgcctaattaattagtcttaattaaatttcaccatgtcaactaagcatttaaaatttaaaacaatttaaattgatcgaataattaacttatttatttatttatgtaaatattaagattcaaattttgttttatatatataataatttattgatttaatgacaaattcttaaataaaatttaaatctatttatgataaattatttttacgataatactagaaaaaaaacaattagaacatactttatttaatatttattaattattgcaacaattgataaatactaaataatataaatttttactgtatttgactaattttttttattactgaatatttttgttatttttacttacgttaccatagtttgcatgcgcacacacatgtatatatatggtgaaaactcaagtgaagttgatacctgagagccgttaaatgatttgactgatttgattagattttcatccaacaactcttaggtatcaactttacgtaaagtcgacttcgcctgagttttcactcatatatatacttgctaagcattattaattttttttcatttttatatatatacactttccatataattttgcgcacttgttttttcttttttgacatgatatacataggtacattttgagaaaaataaggagttttgattagtgtatataaaagaggccttcgtcgtatgcaaagaatatatatatgtgagcgagccaacatgtaaacagtggtgaatgacaaacttttttcaaagaaaagagagaggacccgcatggcaatagcacgcaaattaaataatgatgccttcaattctgatttctgagtacaaaagaaaaattagccaattaataagaaccaattcagatggagacaatattgtaccttgtgagaattagagggatatgatttatttgcgtcataaataacaacatcttcaacttatatatattaaagtttatacacgaaatctcaacttgtttacgtaataatgtattaattatttatatattttatcaaatatattaaattattaaaatcattttaaacttgaaagatcacttttgtttgagttattttaaagaaagaatgtatcttttattgaaaaattttaaataaattaaaataaaataaaataacttctattttaaaaaattaaatttaaaatttaaattcattataaaataataaactcaacaaaaaatcaatatttttaataagtaaaaagggtcattatgaaaataataataaatcctatatgataaaattgaagtgtgaaaaaagaaaatctttgaagaagaagacgatgatgatatcatttaggttgtaaagagtaaaaagatgaaaaaagatatttgtggtggtgacgatgatagcaataataaattaataataatagcaaattaaatagtaacttaaaaaaaatttcaaaaaatgaagagataagaaaaaaaaaacataaaaaattcagaagggttttagtggcattttatttataattgatcaacgtcttattagttttgtcaaatatattgtattttttatgaatattacattagtttaatttattatagttaaatttgtcagaaataaaatttttcatggttaaaacttagaaacggtaatttatctatttattcatgcactactaatctactagctagtatcctttttagcaaaagtttcttttcttgtagcttcatttgtttaaatagtttgtttttcctatagatagttttttttttgttaccatcactagatagagaaaatataaatattgtaaacagattaaaaaaataaatattcaaaaaaagcagataaacaacttaaaattattattattaaaattaataatataaaaaagtctttattattattattattattattattattattattattattattattattattattattattattattattattattattattattattattattattatttggtcctgaaattaaaacattatcgtttaatttatacttgtgtcattccctccaagacttctttcctctatttatttattttttgaatataaagattaaagaacatgagcaccacaaactttttaaagataagaaaagaaaaataaaaaataaaagtgacaagtgacctttaagagtattataaataggtggatactacaatgaagatggcataattgtcttcatatgagtatatttctttttgacctttggatgatggattgtatggttagattttgatatttataaatgtgttgtttttgtttaaagtgtggctaaataaataaaccacacttttaactaaagcatcttcatgagaagatatttttgccatcttcattgtagtatccaccaaataaatatggatgtagagatcacactcccattatacccaacaacaaacaaaattcacttcatattcttcttcttctctttcattctctttaaca

SEQ ID NO: 15 - DNA, 23 bp, Artificial sequence
  Sequence is synthesized
acgaaatctcaacttgtttacgt

SEQ ID NO: 16 - DNA, 23 bp, Artificial sequence
  Sequence is synthesized
tcttccaagaactaaaggatctg

SEQ ID NO: 17 - DNA, 41 bp, Artificial sequence
  Sequence is synthesized
ctcgagctgcaggagctcatggcaaagatgtcatcttcaga

SEQ ID NO: 18 - DNA, 43 bp, Artificial sequence
  Sequence is synthesized
cgaacgaaagctctagacttagcgtcttctagcggcggtttct

SEQ ID NO: 19 - DNA, 31 bp, Artificial sequence
  Sequence is synthesized
cggaattcacgaaatctcaacttgtttacgt

SEQ ID NO: 20 - DNA, 30 bp, Artificial sequence
  Sequence is synthesized
aactgcagtgttaaagagaatgaaagagaa

SEQ ID NO: 21 - DNA, 20 bp, Artificial sequence
  Sequence is synthesized
ggcctagtggtaagagcaga

SEQ ID NO: 22 - DNA, 20 bp, Artificial sequence
  Sequence is synthesized
tgtaaccccaccaagttcaa

(19) United States
(12) Patent Application Publication
(10) Pub. No.: US 2022/0307041 A1
(43) Pub. Date: Sep. 29, 2022

(54) CLONING AND USE OF ARACHIS HYPOGAEA L. FLOWERING HABIT GENE AhFH1 AND ALLELIC VARIANTS THEREOF

(71) Applicant: Qingdao Agricultural University, Qingdao (CN)

(72) Inventors: Xiaojun ZHANG, Qingdao (CN); Jihua LI, Qingdao (CN); Rui GUO, Qingdao (CN); Xiaona YU, Qingdao (CN); Tong SI, Qingdao (CN); Xiaoxia ZOU, Qingdao (CN); Yuefu WANG, Qingdao (CN); Minglun WANG, Qingdao (CN); Xiaoyuan CHI, Qingdao (CN); Shanlin YU, Qingdao (CN)

(73) Assignee: Qingdao Agricultural University, Qingdao (CN)

(21) Appl. No.: 17/617,601

(22) PCT Filed: Oct. 19, 2020
(86) PCT No.: PCT/CN2020/118264

§ 371 Date: Dec. 9, 2021

(30) Foreign Application Priority Data
Jul. 3, 2020 (CN) 202010631475.6

(51) Int. Cl. C12N 15/82

(52) U.S. Cl. CPC C12N 15/827

(57) ABSTRACT

Cloning and use of an Arachis hypogaea L. flowering habit gene AhFH1 and allelic variants thereof are provided. Through experiments, the Arachis hypogaea L. flowering habit gene AhFH1 and two defunctionalized allelic variants thereof are determined. The defunctionalized allelic variants can cause the change from alternate flowering Arachis hypogaea L. to continuous flowering Arachis hypogaea L. Through overexpression of the gene AhFH1 or supplementary expression of a promoter of the gene itself, a continuous flowering Arachis hypogaea L. variety can be changed into alternate flowering Arachis hypogaea L.; and through knockout or expression suppression of the gene AhFH1, alternate flowering Arachis hypogaea L. can be changed into continuous flowering Arachis hypogaea L. Marker-assisted selection (MAS) breeding is realized for allelic variants of the gene using molecular markers.

### TECHNICAL FIELD

The present disclosure belongs to the technical field of plant molecular genetics and genetic engineering, and relates to cloning and use of an Arachis hypogaea L. flowering habit gene AhFH1 and allelic variants thereof. In the present disclosure, through molecular biotechnology, the gene is used to conduct biological genetic improvement for Arachis hypogaea L. flowering habit-related alternate/continuous flowering and Arachis hypogaea L. traits caused thereby such as shoot number, pod number, pod concentration, maturity consistency, and pod yield, or conduct molecular breeding of Arachis hypogaea L by molecular biological means.

### BACKGROUND

Peanuts (Arachis hypogaea L.) are rich in fat, protein, and various vitamins, and have extremely high nutritional value. Therefore, Arachis hypogaea L. has become an important economic crop in many countries (Wan Shubo, 2003). An Arachis hypogaea L. plant has a bunching plant architecture composed of an upright main stem, a pair of first embryo-derived lateral shoots, multiple primary lateral shoots developed at a base of the main stem, and secondary, tertiary, and subprime lateral shoots developed on the lateral shoots. The flowering habit of Arachis hypogaea L. is an important trait related to an Arachis hypogaea L. plant architecture, which specifically refers to differentiation of an axillary bud primordium of Arachis hypogaea L. into inflorescences or shoots, and is finally manifested as the arrangement of inflorescences and lateral shoots on a shoot. There are two main typical representatives: continuous flowering type (FIG. 1A) and alternate flowering type (FIG. 1B). Most typical traits for the continuous flowering type: there are flowers on the main stem and an inflorescence grows at each node (leaf axil) on a lateral shoot, or a secondary vegetative shoot and an inflorescence grow at the basal 1 and 2 nodes or the first node on a primary lateral shoot and inflorescences continuously grow on all subsequent nodes; and inflorescences grow on the first and second nodes and all subsequent nodes on a secondary lateral shoot. This mode limits the shoot number of Arachis hypogaea L., and thus the continuous flowering type is also called sparsely-branched Arachis hypogaea L. Typical traits for the alternate flowering type: there is no flower on the main stem and an inflorescence and a vegetative shoot grow alternately on a lateral shoot, and an alternate growth mode is generally as follows: vegetative shoots grow at the basal 1 to 3 or 1 to 2 nodes on the lateral shoot without inflorescences, and inflorescences grow at the subsequent 4 to 6 or 3 to 4 nodes without vegetative branches (commonly, 2 inflorescences : 2 shoots). As vegetative shoots start to grow from the base and shoots occupy nearly half of nodes, the alternate flowering type has many densely-distributed shoots and thus is also called densely-branched Arachis hypogaea L. In taxonomy, Arachis hypogaea L. cultivars are simply divided into two subspecies based on whether there are flowers on the main stem: continuous flowering subspecies (Arachis hypogaea subsp. fastigiata) and alternate flowering subspecies (Arachis hypogaea subsp. hypogaea) (Krapovickas A, et al. 2007). In addition, there are few atypical or intermediate varieties, which are mainly characterized by continuous flowering and occasional branching, alternate branching and flowering at all leaf axils, and so on. The flowering habit of Arachis hypogaea L. directly affects the aboveground plant architecture of Arachis hypogaea L. by affecting the inflorescence number, shoot number, pod concentration, and maturity consistency of Arachis hypogaea L., which further affects the yield, planting mode, and kernel quality of Arachis hypogaea L. Because leaf axils of Arachis hypogaea L. develop gradually, for continuous flowering Arachis hypogaea L., flowering and podding are relatively concentrated in both time and space, and pods are matured consistently, easy to harvest, and consistent in quality; and for alternate flowering Arachis hypogaea L., there is a large time and space gap between flowering and podding, such that pods are scattered and matured inconsistently, and the yield and quality of pods are compromised.

At present, in plants such as Arabidopsis thaliana (A. thaliana), Lycopersicon esculentum, Glycine max L., Oryza sativa L., and Zea mays L., great progress has been made in the basic genetic research of inflorescence differentiation and development. The inflorescence branching pattern of A. thaliana is regulated through mutual antagonism of the TFL1 homologous gene and the FT homologous gene LFY/AP1 (Alvarez J, 1992; Liljegren S J, 1999; Conti L, 2007; and Hiraoka K, 2013). An A. thaliana tfl1 mutant leads to a solitary flower trait and causes the apical meristem of an inflorescence shoot to stop proliferation to form a single flower (Alvarez J, 1992); and the overexpression of TFL1 causes late flowering in A. thaliana (Benlloch R, 2007). Glycine max L. is a photoperiod-sensitive short-day plant (ADP). Studies have shown that GmFT1a, a member of the Glycine max L. FT gene family, can delay the flowering and maturation of Glycine max L., which plays an antagonistic role with the flowering-promoting gene GmFT2a/GmFT5a to jointly regulate the growth and development of Glycine max L. (Liu W, 2017, Kong F., 2010, Sun H, 2011, and Cai Y, 2018). Zea mays L. is also a typical ADP. Researchers have cloned a quantitative trait locus (QTL) (ZmCCT9) that controls the flowering stage of Zea mays L. through map-based cloning and correlation analysis, and a Harbinger-like transposon upstream of the QTL inhibits the expression of the gene ZmCCT9, thereby promoting the flowering of Zea mays L. under long-day conditions (Huang C, 2017, Hsiao-Yi H, 2012, and Yang Q, 2013). Studies in Lycopersicon esculentum have shown that a ratio of local FT (especially SFT) to TFL1 (SP) controls the balance of limited or unlimited growth of primary and secondary shoots. A branch structure of Lycopersicon esculentum changes with the addition and deletion of florigen (sft) and florigen inhibition genes (sp). Therefore, the use of hybridization and gene editing methods to cultivate high-yield Lycopersicon esculentum varieties provides a new research direction for acquisition of ideal plants (Krieger U., 2010, and Soyk S., 2017).

During the branching development of a plant, the shoot apical meristem (SAM) differentiates into the main stem of the plant, and lateral shoots are differentiated from axillary meristems. The process of differentiation of a lateral shoot from an axillary meristem is regulated jointly by the environment and the plant internal factors. Many genes related to the control of branching development have been obtained in the study of branching patterns of plants such as A. thaliana, Oryza sativa L., Lycopersicon esculentum, and Zea mays L. (Zhi W N T, 2014, and Soyk S., 2017). According to branching phenotypes of plants, these genes can be divided into two categories. One category involves genes for controlling the formation of leaf axillary meristems, such as gene LS first found in Lycopersicon esculentum, which can not only control the formation of axillary meristems, but also cause an is mutant Lycopersicon esculentum plant to have almost no shoot (Schumacher K, 1999); homologous gene OsMOC1 of LS found in Oryza sativa L., whose mutation causes Oryza sativa L. to fail to form tiller buds, thereby affecting the number of Oryza sativa L. tillers (Li X, 2003); gene BL for regulating axillary meristems in Lycopersicon esculentum (Gregor S, 2002); homologous gene RAX of Lycopersicon esculentum BL found in A. thaliana (Keller T, 2006); and so on. The other category involves genes related to the growth of axillary meristems, which mutation do not affect the formation of axillary meristems and include: gene TB1 in Zea mays L. that inhibits the growth of axillary buds (Doebley J, 1997, and Lauren H, 2002); homologous genes of TB1, such as OsTB1 or FINECULM1 in Oryza sativa L. and BRC1 in A. thaliana, Pisum sativum L., and Lycopersicon esculentum (Aguilar-Martinez J A, 2007; Nils B, 2012; Mar M T, 2011; and Minakuchi K, 2010); gene BRC1 in A. thaliana that encodes a similar protein to TB1 and regulates the development of A. thaliana axillary buds (Aguilar-Martinez J A, 2007); and genes S1BRC1a and S1BRC1b found in Lycopersicon esculentum that have similar functions to gene BRC1 in A. thaliana (Mar M T, 2011); and so on. Recently, it has also been reported that strigolactone (SL) regulates the development of lateral shoots of A. thaliana via BRC1 (Wang, L., et al., 2020).

At present, there is little research effort on an Arachis hypogaea L. flowering habit gene. Although bioinformatics analysis has been conducted on the florigen gene family of Arachis hypogaea L., at least 29 members of the florigen homologous gene family of cultivated Arachis hypogaea L. are predicted, and thus it is not clear which gene controls the alternate and continuous flowering of Arachis hypogaea L. (Jin, Tang et al., 2019). There are no reports on the cloning and functional research of an Arachis hypogaea L. flowering habit gene. The mapping and cloning of an Arachis hypogaea L. flowering habit gene will provide a target gene for genetic improvement on the Arachis hypogaea L. flowering habit and related traits and for genetic engineering or gene editing breeding.

### SUMMARY

In order to overcome the above shortcomings, the present disclosure provides cloning and use of an Arachis hypogaea L. flowering habit gene AhFH1 and allelic variants thereof.

In order to achieve the above objective, the present disclosure adopts the following technical solutions:

The present disclosure provides cloning and use of an Arachis hypogaea L. flowering habit gene AhFH1 and allelic variants thereof. Through linkage mapping, map-based cloning, and sequence difference analysis of candidate genes between parents, a genetic segregation population constructed by the hybridization of an alternate flowering Arachis hypogaea L. variety with a continuous flowering Arachis hypogaea L. variety is used to identify a candidate gene AhFH1 (as shown in FIG. 2). The cloning, comparative analysis, and correlation verification of the gene AhFH1 in germplasm resources show that there are at least three allelic variants of the Arachis hypogaea L. flowering habit gene AhFH1: one fully-functional allelic variant AhFH1 and two defunctionalized allelic variants Ahfh1 (including defunctionalized allelic variants Ahfh1-1 and Ahfh1-2). The present disclosure provides use of the gene AhFH1 and allelic variants and promoters thereof in crop genetic improvement, and preferably in the molecular genetic improvement of an Arachis hypogaea L. flowering habit and Arachis hypogaea L. traits caused thereby such as shoot number, pod number, pod concentration, maturity consistency, and pod yield.

The Arachis hypogaea L. flowering habit gene AhFH1 of the present disclosure has a nucleotide sequence shown in SEQ ID NO: 1 at a genomic level, cDNA corresponding to mRNA transcribed by the gene has a sequence shown in SEQ ID NO: 2, and a protein encoded by the gene has a sequence shown in SEQ ID NO: 3. Representative varieties for the allelic variant AhFH1 include the Arachis hypogaea L. genome sequencing variety Tifrunner, the Zhejiang local variety Xiaohongmao, or the like, and the allelic variant corresponds to the alternate flowering habit of Arachis hypogaea L. A cloning primer pair for the Arachis hypogaea L. flowering habit gene AhFH1 at a genomic level is FH1g-F/R, with nucleotide sequences shown in SEQ ID NOs: 4-5, the primer pair is used to clone in representative varieties, and an electrophoretogram of cloning products is shown in FIG. 3. A cloning primer pair for the Arachis hypogaea L. flowering habit gene AhFH1 at a cDNA level is FH1cd-F/R, with nucleotide sequences shown in SEQ ID NOs: 6-7, the primer pair is used to clone the complete coding frame of the fully-functional allelic variant AhFH1 in cDNA of representative varieties, and an electrophoretogram of cloning products is shown in FIG. 4.

The defunctionalized allelic variant Ahfh1-1 of the present disclosure has a nucleotide sequence shown in SEQ ID NO: 8 at a genomic level. The defunctionalized allelic variant Ahfh1-1 has a 1,492 bp deletion from +1,872 bp to +3,273 bp at a genome-wide gene termini that involves the last exon and most or complete 3′UTR and starts from ATG (the deletion is named the functional molecular marker InDel-1492 bp). Representative varieties for the allelic variant include the genome sequencing variety shitouqi, the local variety Fu Peanut, and the like, and the allelic variant corresponds to the continuous flowering habit of Arachis hypogaea L.

The defunctionalized allelic variant Ahfh1-2 of the present disclosure has a nucleotide sequence shown in SEQ ID NO: 11 at a genomic level, and cDNA encoded by the defunctionalized allelic variant Ahfh1-2 has a sequence shown in SEQ ID NO: 12 and has a base C deletion at +335 bp. The base C deletion causes translation frame frameshift of Ahfh1-2 to form a terminator in advance and thus makes a translated protein incomplete and non-functional. Representative varieties for the allelic variant include the Arachis hypogaea L. variety Yunnan Qicai, the Long Peanut 559, and the like, and the allelic variant corresponds to the continuous flowering habit of Arachis hypogaea L. There are a cloning primer pair FH1g-F/R (SEQ ID NOs: 4-5) at a genomic level and a cloning primer pair FH1cd-F/R (SEQ ID NOs: 6-7) at a cDNA level for the above gene AhFH1, which can be used for cloning of Ahfh1-2 at the genomic level and the cDNA level, respectively. Single nucleotide polymorphisms (SNPs) between the gene AhFH1 and the allelic variant Ahfh1-2 can be identified by sequencing for amplification products.

The present disclosure also provides a functional molecular marker InDel-1492 bp for distinguishing the alternate flowering allelic variant AhFH1 and the continuous flowering allelic variant Ahfh1-1 of the Arachis hypogaea L. flowering habit gene, and a corresponding primer pair is InDel-1492 bp-F/R, with nucleotide sequences shown in SEQ ID NOs: 9-10 (this primer pair is a preferred primer pair, and another primer pair that can be used to amplify and identify the above-mentioned 1,492 bp deletion between AhFH1 and Ahfh1-1 can also be used). When it is confirmed in combination with sequencing that there is no mutation in an AhFH1 sequence, amplification products of the functional molecular marker InDel-1492 bp can be used to distinguish the two allelic variants AhFH1 and Ahfh1-1 through agarose electrophoresis. An amplification product of AhFH1 is of 2,556 bp and an amplification product of Ahfh1-1 is of 1,064 bp (FIG. 5).

The present disclosure also provides use of a promoter sequence of the Arachis hypogaea L. flowering habit gene AhFH1 in crop genetic improvement, and preferably in the improvement of an Arachis hypogaea L. flowering habit and traits related thereto such as shoot number, pod number, pod concentration, maturity consistency, and pod yield. There are mainly two promoter sequences shown in SEQ ID NOs: 13-14 for the gene AhFH1/Ahfh1, which are from Tifrunner and shitouqi, respectively. A primer pair FH1p-F/R for cloning the promoter is also provided, with nucleotide sequences shown in SEQ ID NOs: 15-16, and the primer pair can be used to clone the promoter of the gene AhFH1. According to comparison between Tifrunner and shitouqi, the latter mainly has a 214 bp insertion (which is named the molecular marker InDel-214 bp), and this difference can be detected by agarose electrophoresis (FIG. 6). For the representative varieties, three band patterns can be obtained, where in addition to the single short band pattern of Tifrunner and the single long band pattern of shitouqi, there is also a double band pattern of Florunner with both long and short bands. In the double band pattern, one of two subgenomic homologous genes of subgenes A and B of allotetraploid Arachis hypogaea L. has no 214 bp insertion, and the other one has 214 bp insertion. The molecular marker InDel-214 bp can be used for marker-assisted selection (MAS) of the AhFH1 gene locus in the offspring of biparental cross.

The present disclosure also provides an overexpression recombinant construct, where a 35S promoter of tobacco mosaic virus (TMV) is used to construct the overexpression vector p35S::AhFH1 carrying a nucleotide sequence related to the Arachis hypogaea L. flowering habit gene AhFH1, with a plant overexpression vector PHB as a vector backbone; the construction of the overexpression vector requires a primer pair of OE-FH1-F and OE-FH1-R, with sequences shown in SEQ ID NOs:17 -18; the primer pair is used to amplify in cDNA of alternate flowering Arachis hypogaea L. or a plasmid carrying a complete coding frame of the gene to obtain the gene AhFH1, and an amplification product is introduced into the overexpression vector PHB (as shown in FIG. 7) or another plant overexpression vector through enzyme digestion or recombination to construct the overexpression transgenic vector p35S::AhFH1 (as shown in FIG. 7A); and the overexpression vector is transformed into continuous flowering Arachis hypogaea L. to increase the shoot number of the Arachis hypogaea L., thereby affecting other related traits.

The present disclosure also provides a complementary expression recombinant construct, where on the basis of the overexpression transgenic vector p35S::AhFH1 constructed above, a promoter of the gene AhFH1 itself is used to construct the complementary expression transgenic vector pFH1::AhFH1, which carries the nucleotide sequence related to the Arachis hypogaea L. flowering habit gene AhFH1; the construction of the complementary expression vector requires a primer pair of FH1pro-F/R, with sequences shown in SEQ ID NOs: 19-20, where an upstream primer FH1pro-F has an EcoR I restriction site of “gaattc” and a downstream primer FH1pro-R has a Pst I restriction site of “ctgcag”; the primer pair is used to clone a promoter of DNA of an alternate flowering Arachis hypogaea L. variety, an amplification product or a T vector carrying the amplification product is directly digested with EcoR I and Pst I, and then a target fragment is recovered and ligated with a large fragment recovered after the overexpression transgenic vector p35S::AhFH1 undergoes the same digestion linearization to construct the complementary expression transgenic vector pFH1::AhFH1 (as shown in FIG. 7B). In addition, the complementary expression vector can also be constructed as follows: using appropriate primers to directly amplify a full-length genome including a promoter and a coding region of the functional AhFH1 in an alternate flowering variety, and introducing an amplification product into an appropriate plant transgenic vector, which will not be described in detail here. The complementary expression vector, when transformed into continuous flowering Arachis hypogaea L., can change the continuous flowering Arachis hypogaea L. into alternate flowering Arachis hypogaea L. and increase the shoot number, thereby affecting other traits related thereto.

The present disclosure also provides a gene editing vector construct carrying a partial nucleotide sequence of the gene AhFH1 or an allele Ahfh1 according to the present disclosure, where the gene editing vector is named KO-AhFH1; there are preferably two target sequences for the construction of the gene editing: sgRNA1 and sgRNA2, which are shown in SEQ ID NOs: 21-22; one of the two fragments is ligated into an sgRNA region of a CRISPR/Cas9 vector BGKO41 (FIG. 8) to construct the gene editing knockout vector KO-AhFH1 for the target gene AhFH1; the gene editing vector is transformed into an alternate flowering Arachis hypogaea L. variety to change the gene AhFH1 through gene editing, and then defunctionalized offspring individuals are screened out to realize the change from alternate flowering Arachis hypogaea L. to continuous flowering Arachis hypogaea L., which reduces the root number and increases the flower number, the pod number, and other related traits; and the sgRNA1 and sgRNA2 are preferred target sequences, and a different target sequence can be used according to a different CRISPR/Cas9 vector system or editing efficiency.

The Arachis hypogaea L. flowering habit gene AhFH1 and allelic variants thereof according to the present disclosure may be directly derived from Arachis hypogaea L., and may also be derived from homologous gene with sufficiently high similarity in Glycine max L., Brassica napus L., Gossypium spp., Oryza sativa L., Zea mays L., Triticum aestivum L., or other crops.

The present disclosure also provides a method for improving traits related to the Arachis hypogaea L. flowering habit, and the method includes cultivating Arachis hypogaea L. plants with a construct carrying a nucleotide sequence related to the above-mentioned gene AhFH1 or an allele Ahfh1.

The present disclosure has the following beneficial effects.

The cloning and use of an Arachis hypogaea L. flowering habit gene AhFH1 provided in the present disclosure has the following beneficial effects:

(1) The Arachis hypogaea L. flowering habit gene AhFH1 and allelic variants thereof provided by the present disclosure provide important references for exploring a molecular mechanism of the Arachis hypogaea L. flowering habit gene AhFH1 to regulate the Arachis hypogaea L. flowering habit, preliminarily constructing a molecular network of the gene to participate in the regulation of flowering and branching, and studying an evolution law of a function of the gene in crops.

(2) The difference between the Arachis hypogaea L. flowering habit gene AhFH1 and allelic variants thereof provided by the present disclosure can be developed into a functional molecular marker, which can be used for MAS breeding of crops, and preferably plays a key role in the improvement of the Arachis hypogaea L. flowering habit and related traits such as shoot number, pod number, pod concentration, maturity consistency, and pod yield.

(3) A gene sequence and an amino acid, a polypeptide, or protein of the Arachis hypogaea L. flowering habit gene AhFH1 provided by the present disclosure can be used in crop genetic improvement, and preferably play a key role in the improvement of the Arachis hypogaea L. flowering habit and related traits such as shoot number, pod number, pod concentration, maturity consistency, and pod yield.

(4) An overexpression vector, a complementary expression vector, and a gene editing vector carrying the Arachis hypogaea L. flowering habit gene AhFH1 provided by the present disclosure and plants with the vector preferably play a key role in the improvement of the Arachis hypogaea L. flowering habit and related traits such as shoot number, pod number, pod concentration, maturity consistency, and pod yield.

### BRIEF DESCRIPTION OF THE DRAWINGS

FIG. 1 shows a pattern of the Arachis hypogaea L. flowering habit according to the present disclosure, where A represents a continuous flowering type and B represents an alternate flowering type.

FIG. 2 shows a map-based cloning process of the Arachis hypogaea L. flowering habit gene AhFH1 according to the present disclosure.

FIG. 3 is an electrophoretogram for the full-length cloning of the Arachis hypogaea L. flowering habit gene AhFH1 in representative Arachis hypogaea L. varieties at a genomic level (primer pair FH1g-F/R) according to the present disclosure.

FIG. 4 is an electrophoretogram for the cDNA cloning of the Arachis hypogaea L. flowering habit gene AhFH1 (primer pair FH1cd-F/R) according to the present disclosure.

FIG. 5 is an electrophoretogram for the functional molecular marker InDel-1492 bp for distinguishing the two allelic variants AhFH1 and Ahfh1-1 (primer pair InDel-1492 bp-F/R) according to the present disclosure.

FIG. 6 is an electrophoretogram for the cloning of two promoters of the Arachis hypogaea L. flowering habit gene AhFH1 in a genome (primer pair FH1p-F/R) according to the present disclosure.

FIG. 7 is a structural diagram of the constructs p35S::AhFH1 and pFH1::AhFH1 according to the present disclosure.

FIG. 8 is a structural diagram of the gene editing construct KO-AhFH1 according to the present disclosure.

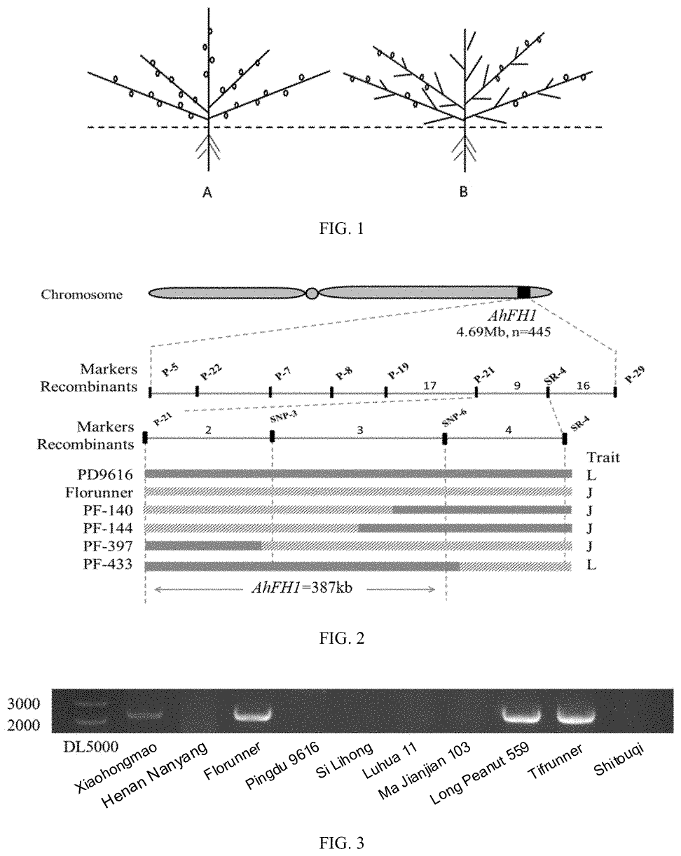

### DETAILED DESCRIPTION OF THE EMBODIMENTS

The technology of the present disclosure is further described below through examples in conjunction with accompanying drawings. Unless otherwise specified, the molecular biology methods used in the examples such as DNA extraction, RNA extraction, reverse transcription from RNA to cDNA, PCR amplification, and enzyme digestion and ligation are conventional molecular biology methods, which can be seen in the “Molecular Biology Experimental Guide”.

### EXAMPLE 1

### Map-Based Cloning of the Arachis hypogaea L. Flowering Habit Gene AhFH1

(1) Construction of Arachis hypogaea L. variety materials and hybrid populations: 268 Arachis hypogaea L. germplasm resources were deposited in the Arachis hypogaea L. Center of Qingdao Agricultural University. The alternate flowering cultivated Arachis hypogaea L. Florunner and the continuous flowering cultivated Arachis hypogaea L. Pingdu 9616 were selected and crossbred to obtain a hybrid population F₁, members of the hybrid population were inbred to obtain a segregation population F₂, and the inbreeding was conducted continuously for multiple generations to finally obtain a recombinant inbred line PF-F₆ of the F₆ generation. The alternate flowering cultivated Arachis hypogaea L. Xiaohongmao and the continuous flowering cultivated Arachis hypogaea L. Henan Nanyang were crossbred to obtain a hybrid population F₁, members of the hybrid population were inbred to obtain a segregation population F₂, and the inbreeding was conducted continuously for multiple generations to finally obtain a recombinant inbred line HN-F₇ of the F₇ generation.

(2) Extraction of plant DNA: The improved CTAB method was used to extract plant DNA.

(3) Extraction and reverse transcription of plant RNA: The RNA Extraction Kit from Takara was used to extract RNA, and the PrimeScript™ RT reagent Kit from Takara was used for reverse transcription of the RNA into cDNA.

(4) Use of the Advanced-BSR-Seq (Advanced Bulked Segregation RNA sequencing) method to initially map the Arachis hypogaea L. flowering habit gene AhFH1 (with reference to Patent CN110675915A “Method for Simultaneously Mapping Two Trait-related Genes”):

Transcriptome sequencing was conducted for Pingdu 9616, Florunner, and 60 offspring individuals (30 alternate flowering individuals and 30 continuous flowering individuals) to obtain transcriptome sequencing data of 62 samples. SNP results were screened through alignment of the transcriptome data with the reference genome sequence of the cultivar Tifrunner to finally obtain 12,421 high-quality and credible SNP loci. The high-quality SNPs were subjected to SNP-index analysis between an alternate flowering pool and a continuous flowering pool, and the flowering habit gene was initially mapped at an end of chromosome 12 (namely, between 117,682,534 bp and 119,846,824 bp on chromosome 12), with a total length of about 2.16 M (Tifrunner Reference Genome, first edition).

(5) Fine mapping of the Arachis hypogaea L. flowering habit gene AhFH1 and prediction of candidate genes:

The whole population of the recombinant inbred line constructed from the continuous flowering Arachis hypogaea L. variety Pingdu 9616 and the alternate flowering Arachis hypogaea L. variety Florunner was used to map the gene for controlling the Arachis hypogaea L. flowering habit between InDel markers P-21 and P-29 at an end of chromosome 12 (with a length of about 0.89 Mb) through linkage mapping. Further, 25 recombinant individuals (population consisting of 445 strains) obtained from the linkage mapping verification between the markers P-21 and P-29 were further subjected to genotype identification with the internal InDel markers, and in combination with phenotype analysis, the target locus was mapped in the narrowed 446 kb interval between InDel markers P-21 and SR-4. Based on the resequencing data of parental genomes, multiple sequencing fragments were designed in this interval for parental sequencing, 2 SNP markers were obtained between the parents, and the internal 9 recombinant individuals were subjected to sequencing and phenotype comparison, such that the locus was finally mapped in the narrowed 387 KB interval between P-21 and SNP-6 (see FIG. 2, the labeled primers were shown in Table 1). In this 387 KB interval, about 44 genes were predicted and annotated in the reference genome. Through bioinformatics analysis, a gene Arahy.BBG51B of the PEBP/CET gene family (florigen FT gene family) in the interval was preliminarily determined as a candidate gene for the Arachis hypogaea L. flowering habit gene AhFH1.

TABLE 1
Information of primers for markers of fine mapping

| Marker | | |
|---|---|---|
| name | Primer name | Primer sequence |
| P-21 | P-21-F | 5′ AAATAATAAAGCCGCTTTAGG 3′ |
| | P-21-R | 5′ AAGAAGTTTGTCAACCATCCC 3′ |
| SNP-6 | SNP-6-F | 5′ AAAATACACCATTAATCACCTTTG 3′ |
| | SNP-6-R | 5′ GATGATGTAAAAGAACCAATTTCTA 3′ |
| SR-4 | SR-4-F | 5′ AAACCTACCAAAACCTTTATCAT 3′ |
| | SR-4-R | 5′ ATAGTAAGTAATTCGGACGAACA 3′ |
| P-29 | P-29-F | 5′ TTAAAATTGTTGTCCCTCAAACTT 3′ |
| | P-29-R | 5′ ATGGAATAAGTTAGGTAATGATTCT 3′ |
| SNP-3 | SNP-3-F | 5′ CTTGTTTGAAAGTGCTTGGACTT 3′ |
| | SNP-3-R | 5′ ATTGTTGGAATGAGGATTGGGAT 3′ |

(6) Cloning and correlation verification of the Arachis hypogaea L. flowering habit gene AhFH1:

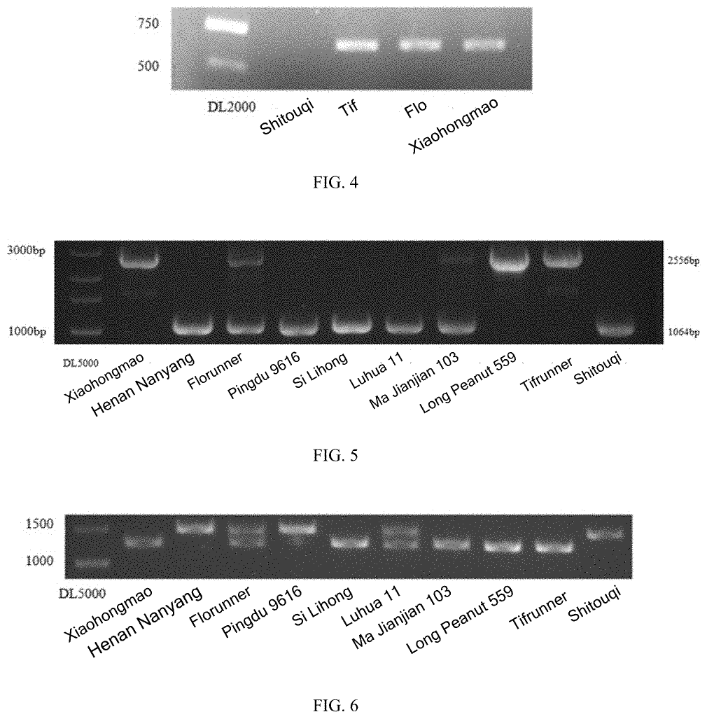

The Arahy.BBG51B was preliminarily determined as a candidate gene of the Arachis hypogaea L. flowering habit gene AhFH1 through fine mapping. The sequence alignment of this candidate gene with the reference genomes of Tifrunner (alternating flowering) and shitouqi (continuous flowering) revealed that there was a 214 bp insertion in a promoter region of the reference genome of shitouqi (continuous flowering), and the reference sequence of the coding region of shitouqi was unknown. According to the reference sequence of Tifrunner, the primer pair FH1g-F/R (SEQ ID NOs: 4-5) for cloning of the gene AhFH1 at a genomic level was designed, and with the genomic DNA of alternate flowering Arachis hypogaea L. as a template, this primer pair was used to clone the complete genomic sequence of the candidate gene through PCR amplification (FIG. 3). The primer pair FH1cd-F/R for cloning the gene AhFH1 from the cDNA was designed according to the reference sequence, which had sequences shown in SEQ ID NOs: 6-7, and with cDNA of a lateral shoot stem apex or leaf tissue of alternate flowering Arachis hypogaea L. as a template, the primer pair was used to clone the complete coding frame of the candidate gene through PCR amplification (FIG. 4). Representative cultivars involved in this example were Xiaohongmao, Henan Nanyang, Florunner, Pingdu 9616, Si Lihong, Luhua 11, Ma Jianjian 103, Long Peanut 559, Tifrunner, and shitouqi. Since the full-length gene at the genomic level failed to be cloned for some representative cultivars in the amplification of the full-length gene (primer pair FH1g-F/R), a primer pair with an increased span was designed for the downstream of the candidate gene, and the primer pair can be used to amplify the allelic variant Ahfh-1 (SEQ ID NO: 8) corresponding to a small fragment. Sequencing of an amplified fragment showed that there was a 1,492 bp deletion. The primer pair was named InDel-1492 bp-F/R, with sequences shown in SEQ ID NOs: 9-10, which can be used to directly identify the two allelic variants AhFH1 and Ahfh1-1. An amplification product corresponding to the allelic variant AhFH1 was of 2,556 bp, an amplification product corresponding to the allelic variant Ahfh1-1 was of 1,064 bp, and a difference therebetween can be detected by agarose electrophoresis (see FIG. 5). The molecular marker can be used to conduct MAS of the flowering habit allelic variant in crossbreeding between varieties of allelic variants AhFH1 and Ahfh1-1, or to identify allelic variants AhFH1 and Ahfh1-1 in germplasm resources.

According to the reference sequence, the primer pair FH1p-F/R (SEQ ID NOs: 15-16) for cloning a promoter was designed, the promoter of the candidate gene AhFH1 was cloned in the representative varieties, and target bands were subjected to sequencing and comparative analysis. Through the cloning, sequencing, and comparative analysis, it was found that there were mainly two promoter sequences for the candidate gene AhFH1, which were from Tifrunner and shitouqi (SEQ ID NOs: 13-14), respectively. However, there were three cloning band patterns for the germplasms, where in addition to the single short band of Tifrunner and the single long band of shitouqi, there was also a double band pattern with both long and short bands (FIG. 6). In combination with the analysis of later research results, it was found that the double band pattern referred to the existence of the two promoters in the two subgenes A and B of allotetraploid Arachis hypogaea L.

Through cloning of the promoter region and gene end, it was found that the sequences on the genomes A and B of the parent Xiaohongmao (alternate flowering) were complete and identical, and there was no promoter insertion and gene end deletion. The sequences of the gene on the genomes A and B of Pingdu 9616 (continuous flowering) and Henan Nanyang (continuous flowering) were also identical, but there were a 214 bp insertion in the promoter and a 1,492 bp deletion at the gene end. However, for the parent Florunner (alternate flowering), two bands were obtained in the amplification of the promoter and gene end, and according to sequencing analysis, the two bands were both target bands. Analysis showed that there was an allelic difference between the genomes A and B, there were a promoter insertion and a gene end deletion on the genome A, and there was no promoter insertion and gene end deletion on the genome B. It can be seen that there was a difference in the genome B between Pingdu 9616 and Florunner. This result also verified that the flowering habit of the RIL population constructed from Xiaohongmao and Henan Nanyang was controlled by two loci, and the flowering habit of the RIL population constructed from Pingdu 9616 and Florunner was controlled by one locus. The promoter region insertion (214 bp) and the gene end deletion (1,492 bp) found in sequencing and comparison of representative cultivated Arachis hypogaea L. varieties may affect the continuous flowering and alternate flowering of Arachis hypogaea L. Therefore, the promoter region insertion (214 bp) and the gene end deletion (1,492 bp) found in the Arachis hypogaea L. flowering habit candidate gene were developed into InDel markers, which were defined as FH1p-F/R and InDel-1492 bp, respectively. Correlation verification was conducted in 268 germplasm resources with abundant flowering habits, and it was found that the promoter region insertion (214 bp) was not highly correlated with the phenotype, germplasms with the gene end deletion (1,492 bp) were all of the continuous flowering type, but many of germplasms without gene end deletion were also of the continuous flowering type. The gene coding region of the gene AhFH1 at the cDNA level was cloned and sequenced for continuous flowering germplasms without deletion, and it was found that cDNA encoded in the fourth exon of the gene AhFH1 in such germplasms had a base C deletion at +335 bp (allelic variant Ahfh1-2 (SEQ ID NO: 11)), which led to the advanced formation of a terminator and thus made a translated protein lack 63 amino acids, thereby affecting the flowering habit of Arachis hypogaea L.

According to whether there is the promoter region insertion (214 bp), the gene end deletion (1,492 bp), or the base C deletion at +335 bp on cDNA encoded in the fourth exon, correlation analysis was conducted for the 268 germplasm resources, and it was found that the phenotype of the 268 germplasm resources was 100% consistent with the defunctionalization or functionalization of AhFH1, where fully-functional AhFH1 corresponded to alternate flowering, defunctionalized Ahfh1-1 or Ahfh1-2 corresponded to continuous flowering, and a hybridization of the subgenomes A and B corresponded to alternate flowering. As a result, the candidate gene Arahy.BBG51B was determined as the Arachis hypogaea L. flowering habit gene AhFH1. The gene AhFH1 had a fully-functional allelic variant AhFH1 and at least two defunctionalized allelic variants Ahfh1-1 and Ahfh1-2.

The reference sequence of the alternate flowering sequencing variety Tifrunner was analyzed, and it was found that the homologous chromosomes A02 and B02 from different sets of chromosomes of the variety were almost identical in the range of about 500 kb upstream and downstream of this candidate gene, which may be caused by translocation between subgenomes A and B; and the Arahy.DYRS20 and Arahy.BBG51B annotated to the genome A02 were exactly the same. Therefore, the AhFH1 described in this example included two loci: Arahy.DYRS20 on chromosome A02 (named AhFH1A) and Arahy.BBG51B on chromosome B02 (named AhFH1B). In combination with the gene cloning and correlation verification analysis, it showed that, in the allotetraploid cultivated Arachis hypogaea L. composed of two subgenomes A and B, the Arachis hypogaea L. flowering habit gene AhFH1 theoretically had four genotypes: AhFH1A/AhFH1B, Ahfh1a/Ahfh1b, Ahfh1a/AhFH1B, and AhFH1A/Ahfh1b. Generally, in the same variety, the A was exactly equal to the B and the a was exactly equal to the b. Therefore, the genotypes could be simply divided into three types: AhFH1/AhFH1, Ahfh1/Ahfh1, and AhFH1/Ahfh1, where AhFH1/AhFH1 and AhFH1/Ahfh1 were alternate flowering genotypes and only Ahfh1/Ahfh1 was a continuous flowering genotype. For the case where two alternate flowering parents were crossbred to obtain continuous flowering individuals, theoretically, varieties of the two genotypes Ahfh1a/AhFH1B and AhFH1A/Ahfh1b were crossbred to obtain offspring individuals with the recombinant Ahfh1a/Ahfh1b, which was corresponding to the continuous flowering phenotype.

### EXAMPLE 2

### Overexpression Trangsgenesis of the Arachis hypogaea L. Flowering Habit Gene AhFH1

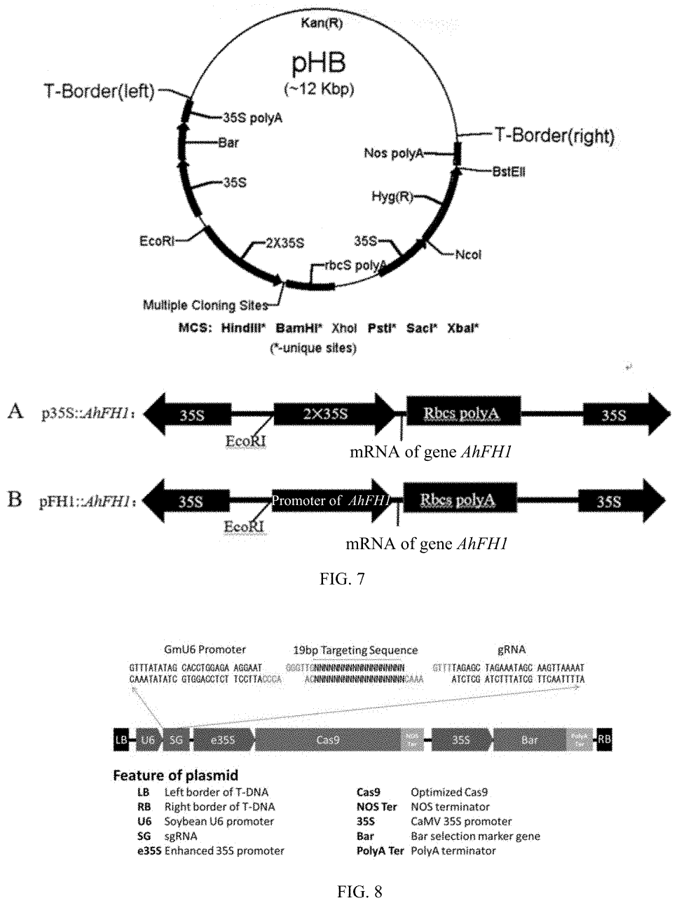

In this example, 35S of TMV was used as a promoter to construct an overexpression transgenic vector p35S::AhFH1, and mRNA of the Arachis hypogaea L. flowering habit gene AhFH1 was overexpressed in a continuous flowering variety (Huayu 23) by the pollen tube introduction method. Specific steps were as follows: GFP on an overexpression vector PHG was cut off through double enzyme digestion with Sac I and Xba I; with a T plasmid as a template, OE-AhFH1-F and OE-AhFH1-R for homologous recombination (with sequences shown in SEQ ID NOs: 17-18) were used to amplify a target fragment; the target fragment amplified from the T plasmid and a backbone fragment of the overexpression vector PHB were recovered and purified through gel, and then ligated through homologous recombination; a ligation product was transformed into competent Escherichia coli (E. coli) DH5a by heat shock, and then the competent E. coli was coated on a LB plate with kanamycin; single colonies were picked for PCR detection, positive colonies were sent to Qingdao Qingke Zixi Biotechnology Co., Ltd. for sequencing, and correct strains were selected for shaking cultivation; a plasmid carrying the target fragment was extracted, which was the AhFH1 overexpression transgenic vector: p35S::AhFH1, with a structure shown in FIG. 7A; the AhFH1 overexpression vector was transformed into competent Agrobacterium tumefaciens (A. tumefaciens) GV3101, then the A. tumefaciens was coated on a YEB plate with kanamycin and rifampicin, and single colonies were picked for PCR detection to obtain positive colonies for later use, which were transgenic strains; and The overexpression transgenic vector, when transformed into continuous flowering Arachis hypogaea L., can increase the shoot number, thereby affecting other traits related thereto.

### EXAMPLE 3

### Complementary Expression of a Promoter of the Arachis hypogaea L. Flowering Habit Gene AhFH1 Itself

The construction of a complementary expression transgenic vector required a primer pair of FH1pro-F and FH1pro-R, with sequences shown in SEQ ID NOs: 19-20. The primer pair was used to clone with DNA of alternate flowering Arachis hypogaea L. as a template, an amplification product or a T vector carrying the amplification product was directly digested with EcoR I and Pst I, and then a target fragment was recovered and ligated with a product obtained after the overexpression transgenic vector p35S::AhFH1 underwent the same digestion linearization to construct the complementary expression transgenic vector pFH1::AhFH1 (as shown in FIG. 7B).

FH1pro-F:
(SEQ ID NO: 18)
5′-CGGAATTCACGAAATCTCAACTTGTTTACGT-3′
FH1pro-R:
(SEQ ID NO: 19)
5′-AACTGCAGTGTTAAAGAGAATGAAAGAGAA-3′;

(FH1pro primers: the upstream AhFH1pro-F had an EcoR I restriction site of “GAATTC” and the downstream FH1pro-R had a Pst I restriction site of “CTGCAG”).

The complementary expression transgenic vector can also be constructed as follows: using appropriate primers to directly amplify a full-length genome including a promoter and a coding region of the functional AhFH1 in an alternate flowering variety, and introducing an amplification product into an appropriate plant transgenic vector, which will not be described in detail here.

A promoter of the Arachis hypogaea L. flowering habit gene AhFH1 itself was used to construct an overexpression vector, and mRNA of the Arachis hypogaea L. flowering habit gene AhFH1 was overexpressed in a continuous flowering variety (Huayu 23) by the pollen tube introduction method. Specific steps were as follows: on the basis of the constructed overexpression vector p35S::AhFH1 of the 35S promoter, the promoter of the gene itself was used to construct an expression vector; the overexpression vector p35S::AhFH1 was digested with EcoR I and Pst I to remove the 35S promoter sequence, and a large fragment (about 12 kbp) of the overexpression vector p35S::AhFH1 was recovered; a promoter of the gene AhFH1 of cultivated Arachis hypogaea L. Xiaohongmao was cloned using a primer pair FH1pro-F/R (with sequences shown in SEQ ID NOs: 19-20) and then ligated with a T vector, a ligation product was transformed, and a resulting plasmid was extracted and sequenced; an extracted plasmid was digested with EcoR I and Pst I, and a target fragment was recovered; the recovered large fragment of the overexpression vector p35S::AhFH1 and the recovered target fragment were ligated by T4 ligase and then transformed into E. coli, and a resulting plasmid was extracted, digested with an enzyme, and sequenced to obtain the complementary expression vector pFH1::AhFH1 for the promoter of AhFH1 itself, with a structure shown in FIG. 7B; and the overexpression vector of the promoter of the AhFH1 itself was transformed into competent A. tumefaciens GV3101, then the A. tumefaciens was coated on a YEB plate with kanamycin and rifampicin, and single colonies were picked for PCR detection to obtain positive colonies for later use, which were transgenic strains. The complementary expression vector, when transformed into continuous flowering Arachis hypogaea L., can change the continuous flowering Arachis hypogaea L. into alternate flowering Arachis hypogaea L. and increase the shoot number, thereby affecting other traits related thereto.

### EXAMPLE 4

### Knockout of the Arachis hypogaea L. Flowering Habit Gene AhFH1 Through Gene Editing

In this example, the CRISPR/Cas9 system was used to conduct knockout through gene editing. Specific operation steps were as follows: an sgRNA target sequence was designed and generated online (http://www.biogle.cn/index/excrispr), and two target sites sgRNA1 and sgRNA2 (SEQ ID NOs: 21-22) with the highest score were selected; a generated sgRNA sequence was used by Qingdao Qingke Zixi Biotechnology Co., Ltd. to synthesize two complementary single-stranded Oligos, and synthesized Oligos were dissolved in water to 10 μM, and 18 μl of Buffer Anneal, 1 μl of Up Oligo, and 1 μl of Low Oligo were mixed in a 200 μl PCR tube, heated at 95° C. for 3 min, and then slowly cooled to 20° C. at a rate of about 0.2° C./s to prepare an Oligo dimer (details can be seen in the BIOGEL vector manual); the Oligo dimer was introduced into a linearized CRISPR/Cas9 vector (which was a KO-AhFH1 vector) by a ligase; 2 μl of the KO-AhFH1 vector, 1 μl of the Oligo dimer, 1 μl of Enzyme Mix, and 16 μl of ddH2O were thoroughly mixed in a 200 μl PCR tube to allow a reaction at room temperature (20° C.) for 1 h; a ligation product was transformed into competent E. coli DH5a by heat shock, and then the competent E. coli was coated on an LB plate with kanamycin; single colonies were picked for PCR detection, positive colonies were sent to Qingdao Qingke Zixi Biotechnology Co., Ltd. for sequencing, and correct strains were selected for shaking cultivation; a resulting plasmid was extracted, which was an AhFH1 knockout plasmid: KO-AhFH1-1/2; the AhFH1 gene knockout plasmid KO-AhFH1-1/2 was transformed into competent A. tumefaciens, then the A. tumefaciens was coated on a YEB plate with kanamycin and rifampicin, and single colonies were picked and subjected to PCR detection; positive colonies were selected and transformed into alternate flowering Arachis hypogaea L. (such as Xiaohongmao or 209 Small Peanut). BGKO41 was used as the CRISPR/Cas9 vector (as shown in FIG. 8). The vector used the Glycine max L. U6 promoter to drive the sgRNA sequence, which can be efficiently used for dicotyledonous plants. An enhanced CaMV 35S promoter was used to achieve the efficient expression of the Cas9 protein. The gene editing vector was transformed into an alternate flowering Arachis hypogaea L. variety to change the gene AhFH1 through gene editing, and then defunctionalized offspring individuals were screened out to realize the change from alternate flowering Arachis hypogaea L. to continuous flowering Arachis hypogaea L., which reduced the root number and increased the flower number, the pod number, and other related traits.

The backbone of the CRISPR/Cas9 vector BGKO41 used for the gene editing was purchased from BIOGLE (http://www.biogle.cn/index/excrispr), which was only used for illustration of examples. Other plant CRISPR/Cas9 gene editing vectors or other single-base editing vectors can also be used.

What is claimed is:

1. A method of using an Arachis hypogaea L. flowering habit gene AhFH1 and allelic variants thereof in crop genetic improvement, wherein the method is configured to improve a flowering habit and traits related to the flowering habit, the traits comprise shoot number, pod number, pod concentration, maturity consistency, and pod yield; the Arachis hypogaea L. flowering habit gene AhFH1 has a nucleotide sequence shown in SEQ ID NO: 1, cDNA encoded by the Arachis hypogaea L. flowering habit gene has a nucleotide sequence shown in SEQ ID NO: 2, and a protein encoded by the Arachis hypogaea L. flowering habit gene has an amino acid sequence shown in SEQ ID NO: 3; there are mainly two promoter sequences shown in SEQ ID NOs: 13-14 for the Arachis hypogaea L. flowering habit gene AhFH1, and the two promoter sequences are from Tifrunner and shitouqi, respectively; allelic variants of the Arachis hypogaea L. flowering habit gene AhFH1 comprise a defunctionalized allelic variant Ahfh1-1 and a defunctionalized allelic variant Ahfh1-2; the defunctionalized allelic variant Ahfh1-1 has a nucleotide sequence shown in SEQ ID NO: 8, has a 1,492 bp deletion from +1,872 bp to +3,273 bp at a genome-wide gene termini involving the last exon and 3′UTR and starting from ATG, and corresponds to a continuous flowering habit of Arachis hypogaea L.;
and the defunctionalized allelic variant Ahfh1-2 has a nucleotide sequence shown in SEQ ID NO:
11, and cDNA encoded by the defunctionalized allelic variant Ahfh1-2 has a sequence shown in SEQ ID NO: 12 and has a base C deletion at +335 bp, causing a cDNA translation frame frameshift to form a terminator in advance and thus making a translated protein incomplete, the defunctionalized allelic variant Ahfh1-2 corresponds to the continuous flowering habit of Arachis hypogaea L.

2. Cloning primers for an Arachis hypogaea L. flowering habit gene AhFH1, specifically comprising: a cloning primer pair FH1g-F/R for the Arachis hypogaea L. flowering habit gene AhFH1 at a genomic level, nucleotide sequences of the cloning primer pair FH1g-F/R are shown in SEQ ID NO: 4 and SEQ ID NO: 5; a cloning primer pair FH1cd-F/R for the Arachis hypogaea L. flowering habit gene AhFH1 at a cDNA level, nucleotide sequences of the cloning primer pair FH1cd-F/R are shown in SEQ ID NO: 6 and SEQ ID NO: 7; and a cloning primer pair FH1p-F/R for two different promoters of the Arachis hypogaea L. flowering habit gene AhFH1, nucleotide sequences of the cloning primer pair FH1p-F/R are shown in SEQ ID NO: 15 and SEQ ID NO: 16, the cloning primer pair FH1p-F/R is configured to identify the two different promoters of the Arachis hypogaea L. flowering habit gene AhFH1.

3. A functional molecular marker InDel-1492 bp for distinguishing a fully-functional Arachis hypogaea L. flowering habit gene AhFH1 and a defunctionalized Arachis hypogaea L. flowering habit gene Ahfh1-1, wherein a corresponding primer pair InDel-1492 bp-F/R has nucleotide sequences shown in SEQ ID NO: 9 and SEQ ID NO: 10; and when it is confirmed in combination with sequencing that there is no mutation in an AhFH1 sequence, the functional molecular marker InDel-1492 bp is configured to directly distinguish the fully-functional Arachis hypogaea L. flowering habit gene AhFH1 and the defunctionalized Arachis hypogaea L. flowering habit gene Ahfh1-1 through PCR and electrophoresis.

4. A construction method and use of an overexpression transgenic vector carrying an Arachis hypogaea L. flowering habit gene AhFH1, wherein a 35S promoter of tobacco mosaic virus (TMV) is configured to construct the overexpression transgenic vector p35S::AhFH1 carrying a nucleotide sequence related to the Arachis hypogaea L. flowering habit gene AhFH1, with a plant overexpression vector PHB as a vector backbone;
the construction method of the overexpression vector requires a primer pair of OE-FH1-F and OE-FH1-R, with sequences shown in SEQ ID NOs: 17-18; the primer pair is configured to amplify in cDNA of alternate flowering Arachis hypogaea L. or a plasmid carrying a complete coding frame of the Arachis hypogaea L. flowering habit gene to obtain the Arachis hypogaea L. flowering habit gene AhFH1, and an amplification product is introduced into the plant overexpression vector PHB or another plant overexpression vector through enzyme digestion or recombination to construct the overexpression transgenic vector p35S::AhFH1; and the overexpression vector is transformed into continuous flowering Arachis hypogaea L. to increase a shoot number of the continuous flowering Arachis hypogaea L., thereby affecting other related traits.

5. A construction method and use of a complementary expression transgenic vector carrying a nucleotide sequence related to an Arachis hypogaea L. flowering habit gene AhFH1, wherein on the basis of the an overexpression transgenic vector p35S::AhFH1, a promoter of the Arachis hypogaea L. flowering habit gene AhFH1 itself is configured to construct the complementary expression transgenic vector pFH1::AhFH1 carrying the nucleotide sequence related to the Arachis hypogaea L. flowering habit gene AhFH1; the construction method of the complementary expression vector requires a primer pair of FH1pro-F/R, with sequences shown in SEQ ID NOs: 19-20, wherein an upstream primer FH1pro-F has an EcoR I restriction site of “gaattc”, and a downstream primer FH1pro-R has a Pst I restriction site of “ctgcag”; the primer pair is used configured to clone and amplify a promoter of DNA of an alternate flowering Arachis hypogaea L. variety, an amplification product or a T vector carrying the amplification product is directly digested with EcoR I and Pst I, and then a target fragment is recovered and ligated with a large fragment recovered after the overexpression transgenic vector p355::AhFH1 undergoes the same digestion linearization to construct the complementary expression transgenic vector pFH1::AhFH1; and the complementary expression transgenic vector pFH1::AhFH1 is transformed into continuous flowering Arachis hypogaea L. to change the continuous flowering Arachis hypogaea L. into alternate flowering Arachis hypogaea L. and increase the a shoot number, thereby affecting other traits related thereto.

6. A construction method and use of a gene editing vector carrying a gene AhFH1 or an allele Ahfh1, wherein the gene editing vector is named KO-AhFH1; there are two target sequences for the construction method of the gene editing vector:
sgRNA1 and sgRNA2 shown in SEQ ID NOs: 21-22; one of the two target sequences is ligated into an sgRNA region of a CRISPR/Cas9 vector BGKO41 to construct the gene editing vector KO-AhFH1 for the gene AhFH1; the gene editing vector is transformed into an alternate flowering Arachis hypogaea L. variety to change the gene AhFH1 through gene editing, and then defunctionalized offspring individuals are screened out to realize a change from alternate flowering Arachis hypogaea L. to continuous flowering Arachis hypogaea L., reducing a shoot number and increases a flower number, a pod number, and other related traits; and the sgRNA1 and the sgRNA2 are preferred target sequences, and a different target sequence is used according to a different CRISPR/Cas9 vector system or editing efficiency.
